SEQUENCE LISTING (4 sequences)

SEQ ID NO: 1 - DNA, 19 bp, Artificial Sequence
  synthetic oligonucleotide primer
ctcttgcttggtggaggtg

SEQ ID NO: 2 - DNA, 26 bp, Artificial Sequence
  synthetic oligonucleotide primer
tgactctgcagaagtgaaagcctggc

SEQ ID NO: 3 - DNA, 19 bp, Artificial Sequence
  synthetic oligonucleotide primer
ctccttggtgatgggagct

SEQ ID NO: 4 - DNA, 20 bp, Artificial Sequence
  synthetic oligonucleotide primer
gtccttgcagatgggatgac

(19) United States
(12) Patent Application Publication
(10) Pub. No.: US 2005/0069881 A1
(43) Pub. Date: Mar. 31, 2005

(54) 3-Hydroxymethylglutaryl coenzyme a reductase and diagnosis and prognostication of dementia

(76) Inventors: Judes Poirier, Boisbriand, QC (CA); Louise Theroux-Lamarre, Varennes (CA); Doris Dea, Verdun (CA); Nicole Aumont, Richelieu (CA)

Correspondence Address: FETHERSTONHAUGH - SMART & BIGGAR, 1000 DE LA GAUCHETIERE WEST, SUITE 3300, MONTREAL, QC, H3B 4W5, CA

(21) Appl. No.: 10/487,747

(22) PCT Filed: Aug. 29, 2002
(86) PCT No.: PCT/CA02/01333

Related U.S. Application Data
(60) Provisional application No. 60/315,346, filed on Aug. 29, 2001.

(51) Int. Cl. C12Q 1/68; C12P 19/34

(52) U.S. Cl. 435/6; 435/91.2

(57) ABSTRACT

The invention relates to methods and commercial packages for the diagnosis and/or prognostication of a dementia. The methods are based upon the assessment of a feature or features relating to 3-hydroxymethylglutaryl coenzyme A reductase (HMGR) gene, a polymorphism associated with the gene, the nature of mRNA transcripts produced, and/or HMGR protein activity. Applicants have determined that a decrease in HMGR activity correlates with Alzheimer disease. Applicants have further determined that the presence of a polymorphism in the HMGR gene correlates with Alzheimer disease. Applicants have identified two abnormal HMGR mRNA transcripts and have shown that their presence correlates with Alzheimer disease.

### FIELD OF THE INVENTION

The invention relates to methods for the diagnosis and prognostication of dementia based on 3-hydroxymethylglutaryl coenzyme A reductase (HMGR) activity and expression.

### BACKGROUND OF THE INVENTION

An example of a dementia is Alzheimer disease (AD). AD is a progressive neurodegenerative disorder with clinical characteristics and pathological features. AD is etiologically heterogenous and can be produced by mutations on genes localized on chromosomes 1, 14 and 21. Major risk factors have been identified for the common form of AD (also refer to as sporadic AD). These include the apolipoprotein E4 allele (chromosome 19), butirylcholinesterase K (chromosome 3), alpha₂-macroglobulin (chromosome 12), lipoprotein lipase (Baum et al., 1999) and two of the apoE receptors called LRP (Beffert et al., 1999a [chromosome 12]) and VLDL receptor (Okuizumi et al., 1995). The apolipoprotein E4 polymorphism was also shown to affect age of onset, rate of progression, cholinergic function and therapeutic response in AD. Several other genetic risk factors have been identified in sporadic AD but replication has proven difficult for those novel risk markers. To date, there is no known genetic mutation responsible for the common form of AD. These observations, combined with several independent genome scans, indicate that AD has a genetic etiology that includes several genetic loci, of which only a minority have been identified so far (Pericak-Vance et al., 1998; Kehoe et al. 1999).

Alzheimer disease (AD) is considered today to be a multifactorial disease with a strong genetic component. It is generally agreed that the disease can be subdivided into two distinct categories: the [so-called] familial and sporadic forms of the disease. The familial form of AD accounts for roughly 10-15% of all cases worldwide, whereas the sporadic form of AD represents 85-90% of the remaining cases and is generally believed to be of late onset, occurring after 65 years of age.

Familial Forms of Alzheimer Disease

Molecular genetic studies have identified several different genetic loci which are believed to be linked to the presence of AD in the general population. The known genetic causes of AD, which include mutation in the amyloid precursor protein gene and two presenilin genes, are rare and account for about 5% of all cases worldwide. These rare mutations are transmitted as autosomal dominant traits in certain families from Europe, North America and Asia. Although a lot has been learned from the familial studies of the mutation in the amyloid protein precursor gene and the presenilin, the molecular mechanism(s) behind the sporadic form of AD is much more complex and requires a different approach. One polymorphism called apolipoprotein E4 on chromosome 19 has been linked to both the late onset familial form, as well as to the sporadic form of AD. The majority of patients referred to as sporadic cases probably arise as the result of several genetic anomalies, each making an independent contribution to the overall phenotype and pathophysiological process. It is suspected that at least one, and most probably several, additional mutations 1, remain to be identified since only 50% of all AD cases have been linked to specific genetic anomalies in case/control studies.

The Amyloid Precursor Protein: The first gene ever identified in association with familial AD was the amyloid precursor protein (APP) (Chartier-Harlin et al., 1991). The APP gene encodes a transcript which, once translated, encodes a single trans-membrane spanning polypeptide of roughly 750 amino acids. Alternative splicing of exon 7 and exon 8 results in a polypeptide of 695 amino acids, which is expressed at very high concentration in the central nervous system. APP is known to undergo a series of proteolytic cleavages which result in the production of a small 40 to 42-amino acid long peptide referred to as the A beta peptide (Sisodia et al., 1990). The exact function of the amyloid precursor protein is currently unknown. Onset at around the age of 50 years is characteristic of familial AD pedigrees associated with mutation in the amyloid precursor protein gene: several mutations (including those at positions 665, 670, 673, 692, 693, 713, 716, 717) have been identified as mutations causing early to late onset familial AD. It has been proposed that these mutations in the APP gene cause an overproduction of the so-called neurotoxic form of beta amyloid referred as the 1-42/1-43 beta peptides. Polymerization of these fibres could then result in the development of senile plaque in the brain of AD patients with a concomitant impact on the brain integrity.

Presenilin 1: Following the discovery that only a portion of the familial cases of AD could be explained by the presence of a mutation in the amyloid precursor protein gene, several independent investigators pursued the hunt for other candidate genes that might be involved in the remaining familial forms of the disease. The presenilin 1 gene localised on chromosome 14 (St-George-Hyslop et al., 1992) was then isolated using positional cloning strategy and more than 35 different mutations were identified by several independent groups as anomalies causing the familial form of the disease. The presenilin 1 gene is transcribed in several organs and in several cell types. There is a concentration of mutations located near or in the highly conserved transmembrane domain of presenilin 1 (Hutton et al., 1996). No deletions, nonsense mutation nor genomic rearrangements have yet been found in the sporadic (common) form of AD. Mutations in the presenilin 1 gene are associated with families where the age of onset is in the late 30s, early 40s.

Presenilin 2: Following the cloning of the presenilin 1 gene on chromosome 14, a very similar sequence has been identified and subsequently localized on chromosome 1 (Levy-Lahad et al., 1995). This polypeptide, referred to as presenilin 2, has an open reading frame of about 448 amino acids with substantial amino acid sequence identity with the presenilin 1 protein. Presenilin 2 appears to be more ubiquitously expressed but less abundant than presenilin 1. It has been proposed that presenilins may be involved in the intracellular trafficking and/or transport of specific proteins inside the cells. Mutational analysis in familial cases of AD uncovered two different missense mutations in the presenilin 2 gene in families segregating for early onset AD.

Sporadic (common) Alzheimer Disease:

Polymorphic genetic markers and the risk of developing Alzheimer disease: The inheritance of common forms of AD appears considerably more complex than familial AD and probably reflects the co-action or interaction of several genes with environmental factors. One gene that is clearly implicated in this form of the disease is that encoding apolipoprotein E (apoE). The E4 allele of apoE, although neither necessary nor sufficient to cause AD, is strongly associated with increased risk, rate of progression and severity of the neuropathology. The effect of apoE4 appears additive such that heterozygotes and homozygotes are, three and eight times more likely, respectively, to be affected than controls. However, the variation at the apoE locus accounts for at most 50% of the genetic variation in liability (Pericak-Vance and Haines, 1995) to develop the disorder and there must be other genetic variants that account for remaining risk.

A number of candidate gene association studies have been performed in sporadic AD since the identification of the apoE locus. Some positive findings have been claimed but none of these have been consistently confirmed. These include alpha₁-antichymotrypsin (Morgan et al., 1997; Schwab et al., 1999), bleomycin hydrolase (Farrer et al., 1998), lipoprotein lipase (Baum et al., 1999) (Brandi et al., 1999) and LRP (Beffert et al., 1999a). These inconsistencies are likely due to a number of factors such as genetic heterogeneity, ethnicity, issues of statistical power, multiple testing and population stratification (Owen et al., 1997).

Therefore, prior to applicants' work presented herein, most of the positive association studies have been essentially based on testing of genes whose candidature is suggested by existing understanding of the pathophysiology of AD.

Apolipoprotein E and Cholesterol Homeostasis in Alzheimer Disease: Apolipoproteins are protein components of lipoprotein particles. The latter are macromolecular complexes that carry lipids such as cholesterol and phospholipids from one cell to another within a tissue or between organs. Some apolipoproteins regulate extracellular enzymatic reactions related to lipid homeostasis while others are ligands for cell surface receptors that mediate lipoprotein uptake into cells and their subsequent metabolism. ApoE is a component of several classes of plasma and cerebrospinal fluid lipoproteins. ApoE was shown to be synthesized and secreted by glial cells, predominantly astrocytes. Neurons appear to contribute very little to steady state levels of apoE in the brain. Several cell surface receptors for apoE are known to be expressed on many of the different cell types that constitute the brain parenchyma. These receptors are members of a single family and include the low-density lipoprotein (LDL) receptor, the very low-density (VLDL) receptor, the apoER2 receptor, the LDL receptor-related protein (LRP), and the megalin/gp330 receptor. The importance of apoE in lipid homeostasis in the brain is underscored by the fact that major plasma apolipoproteins such as apoB and apoAI are not synthesized in the central nervous system.

Early data from animal lesion paradigms such as sciatic nerve crush and entorhinal cortex lesioning indicate that apoE plays a role in the coordinated storage and redistribution of cholesterol and phospholipids among cells within the remodeling area. FIG. 1 illustrates the role of apoE and its major receptors in the transport and recycling of cholesterol from dead or dying neurons to intact neurons undergoing synaptic remodeling and compensatory terminal outgrowth. It was shown that following neuronal cell loss and terminal differentiation in the CNS, large amounts of lipids-are released from degenerating axon membranes and myelin (FIG. 1, #1). In response, astrocytes (FIG. 1, #2) and macrophages synthesize apoE within the lesion to scavenge lipids from both cellular and myelin debris.

During that critical phase, cholesterol synthesis [as monitored by the activity of the 3,3-hydroxy-methylglutaryl-CoA reductase (HMGR)] is progressively repressed in response to a massive increase in intracellular cholesterol concentration through receptor-mediated internalization. It was demonstrated in different cell culture systems that eukaryote cells obtain their cholesterol from two distinct sources: a) it is synthesized directly from acetyl-CoA through the so-called HMGR pathway or, b) it is imported through the apoE/apoB (LDL) receptor family via lipoprotein-complex internalization (for a review see Beffert et al., 1998b). These two different pathways are tightly coupled: i.e. a reduction of cholesterol internalization through the receptor pathway rapidly causes increases in HMGR activity (cholesterol synthesis), whereas inhibition of intracellular cholesterol synthesis induces expression of the LDL receptor and lipoprotein internalization.

Much of the free cholesterol generated during synapse degradation is stored in astrocytes in the CNS and, in macrophages in the PNS where it is eventually reused during PNS regeneration and CNS reinnervation. Following binding of the apoE/lipoprotein complexes with neuronal LDL receptors, the apoE/Lipoprotein/LDL receptor complex is internalized, degraded and the cholesterol released inside neurons where it is used for membrane synthesis and synaptic remodelling (FIG. 1, #3 and #4). The intra cellular rise in cholesterol causes a down-regulation of HMG-COA reductase activity and mRNA prevalence in granule cell neurons undergoing dendritic and synaptic remodeling (FIG. 1, #7).

In humans, three alleles (2, 3, and 4) at a single gene locus on the long arm of chromosome 19 code for the common isoforms of apoE, namely apoE2, apoE3, and apoE4. This allelic heterogeneity gives rise to a protein polymorphism at two positions: residues 112 and 158 on the mature protein. In 1993, the apoE 4 allele was found to be over-represented in groups of both familial and sporadic case's of late-onset AD. The 4 allele frequency was shown to be significantly higher (˜3-fold i.e. 40%-50%) in the Alzheimer population (Corder et al., 1993; Owen et al., 1997; Poirier et al., 1993a; Farrer et al., 1997′). Interestingly, a sharp decline in the prevalence of the 4 allele was observed in very old subjects (>85 years), suggesting the presence of a very late onset form of AD and consistent with the increased risk of coronary heart disease in apoE4 subjects. A meta-analysis of 40 studies representing nearly 30,000 apoE alleles concluded that the E4 allele represents a major risk factor for AD in all ethnic groups, across all ages between 40 and 90 years (Farrer et al., 1997). Interestingly, careful analysis of regenerative markers in the brain of AD subjects indicates that the E4 sub-population clearly show impaired synaptic plasticity and marked loss of regenerative capacity when compared to age-matched controls or apoE3/3 AD subjects, highlighting the crucial role played by cholesterol transport during compensatory remodelling in the CNS (Arendt et al., 1997).

Apolipoprotein E, Cholesterol levels and the Amyloid Hypothesis of AD: While the abnormal processing of the APP into toxic forms of beta amyloid appears to underlie the pathophysiological process that characterizes chromosome 1, 14 and 21 familial cases, the role of apoE as a potent scavenger of beta amyloid in the brain is certainly consistent with this working hypothesis (Beffert and Poirier, 1998; Beffert et al., 1998b; Beffert et al., 1999b). For a while, it was generally believed that mutations in the apoE gene on chromosome 19 and in the APP gene on chromosome 21 represented independent biochemical pathways with similar outcomes; i.e. dementia of the Alzheimer type. However, recent evidence suggests a direct link between these two apparently separate metabolic pathways. ApoE4 allele dosage was shown to modulate the age of onset of AD in families with the amyloid precursor protein (APP) mutation (Farrer et al., 1997; Hardy, 1994). Strittmatter et al. (1993) and Wisniewski et al. (1993) demonstrated that purified, non-reconstituted, human apolipoprotein E binds avidly to beta amyloid fragments in vitro. Furthermore, apo E4 allele dose was shown to positively correlate with the density of beta A4 immunopositive plaques and neurofibrillary tangles in the cortex and hippocampus of AD subjects. Howland et al. (1998) reported decreased processing of APP in gene-targeted APP mice (humanized for beta amyloid and containing the Swedish familial AD mutation) in response to high dietary cholesterol as evidenced by concomitant decrease in secreted APP (alpha and beta), beta amyloid 1-40 and 1-42. The reduction in beta amyloid peptides (1-40 and 1-42) in the brain inversely correlated with increased concentration of brain apoE.

In recent months, there has been a surprising convergence of the beta amyloid cascade hypothesis with the apoE/cholesterol metabolism. It has been shown that breeding of an apoE knockout mouse with an APP transgenic mouse showing amyloid plaques completely abolished amyloid deposition in the hybrid mice, without affecting the steady state levels of beta amyloid 1-40 and 1-42 in the brain (Bales et al, 1997). Expression of the human apoE3 or apoE4 gene in apoE knockout mice drastically reduces beta amyloid deposition in hybrid human apoE/human APP mice (Holtzman et al., 1999).

Cholesterol Synthesis, HMGR activity and beta amyloid Production: Simons and colleagues found that blocking cholesterol synthesis with the HMGR inhibitor simvastatin in absence of external lipoproteins caused a marked inhibition of beta amyloid formation in primary neuronal cell cultures derived from the hippocampus (Simons et al., 1998).

The 3-Hydroxy-3-Methylglutaryl Coenzyme A Reductase: Rate Limiting Step in Cholesterol Synthesis

Mammalian cells, particularly astrocytes and neurons, cultured in vitro synthesize cholesterol at a rate which is inversely proportional to the cholesterol content in the growth medium. Cholesterol requirements of most mammalian cells are met by two separate but interrelated processes. One process is the endogenous synthesis of cholesterol. This synthesis pathway which involves over 20 reactions is regulated primarily by the activity of the 3-Hydroxy-3-MethylGlutaryl Coenzyme A Reductase (HMGR) which catalyzes the formation of mevalonate, the key precursor molecule in the synthesis of cholesterol. The other process involves the utilization of lipoprotein-derived cholesterol following internalization of the lipoprotein bound to its surface receptor (usually, an apoE-rich lipoprotein complex). Cholesterol homeostasis in brain cells is controlled by the perfect balance between cholesterol influx through the apoE receptors pathway and synthesis via the HMGR pathway, the rate limiting step in cholesterol biosynthesis. However, the brain differs significantly from peripheral organs where a multitude of apolipoproteins such as apoB, apoH, apoA1, A2, apoCI and apoCII are playing a pro-active-role in lipid transport and homeostasis. The brain is entirely devoid of apoB (apbE's main back-up system in the blood) and contains only trace amounts of the other apos described above. For some unknown reason, the brain is extremely dependent on apoE and its accessory proteins to deliver and/or produce cholesterol in the intact or injured brain cells.

Under normal circumstances, cholesterol synthesis via the HMGR pathway is required only when lipoprotein internalization by apoE/apoE receptor pathway is insufficient to meet the cholesterol requirement of the cell (Brown et al., 1973; Rodwell et al., 2000). The endoplasmic reticulum-bound HMGR is regarded as the rate limiting step in the synthesis of cholesterol, a critical membrane lipid, precursor of steroid hormones (glucocorticoids and estrogen) and a signaling molecule involved in embryogenesis (Ness and Chambers, 2000). The other shorter form of HMGR localized in the peroxisomal compartment does not appear to play an important role in cholesterol homeostasis and is far more resistant to commonly used HMGR inhibitors such as simvastatin (Aboushadi et al., 2000). In cells grown in excess of cholesterol-rich lipoproteins, the HMGR activity is down regulated in favor of uptake via apoE receptors (Sato and Takano, 1995). A similar process was reported in the PNS and CNS during the acute phase of regeneration that ensue degradation of dead cells following experimental injury (Boyles et al., 1990) (Poirier et al., 1993b; Poirier, 1994).

To maintain cellular cholesterol homeostasis, there exists a rather potent negative feed-back system on the HMGR activity and gene expression which results in decrease in synthesis of cholesterol in response to excess intracellular sterol internalization via the apoE receptors family (Ness and Chambers, 2000). This first and most important feedback regulation of the HMGR activity is through decrease in gene transcription (Reynolds et al., 1984; Chin et al., 1984). The factor that has been shown to regulate the expression of the reductase is the controlled degradation of the HMGR protein (Gardner and Hampton, 1999; Cronin et al., 2000). Lastly, there is evidence from hamster for a modulation in translation efficiency of mRNA for HMGR resulting in decreased or increased reductase protein and activity (Choi and Choi, 2000).

### SUMMARY OF THE INVENTION

The invention provides methods and commercial packages for the diagnosis and prognostication of a dementia based on an HMGR gene, its transcripts, and activity of HMGR protein. In an embodiment, such a dementia is an Alzheimer disease.

Accordingly, the invention provides a method of diagnosing or prognosticating a dementia in a subject, said method comprising: (a) obtaining a sample from said subject, wherein said sample comprises nucleic acid comprising a 3-hydroxy-3-methylglutaryl coenzyme A reductase (HMGR) gene; and (b) determining whether said nucleic acid comprises a polymorphism relative to a corresponding control sample obtained from a control subject; wherein the presence of said polymorphism is used to diagnose or prognosticate a dementia.

In an embodiment, the above-mentioned polymorphism is localized in intron B (also known as intron 2, eg in hamsters) of said HMGR gene.

In an embodiment, the above-mentioned step (b) comprises:
(i) amplifying a nucleic acid sequence comprising said intron B (also known as intron 2, eg in hamsters) by polymerase chain reaction (PCR) to obtain a PCR product;
(ii) digesting said PCR product with a restriction enzyme to obtain a restriction digest product; and (iii) determining a size of said restriction digest product.

In an embodiment, the above-mentioned restriction enzyme is ScrFI.

The invention further provides a method of diagnosing or prognosticating a dementia in a subject, said method comprising: (a) measuring a first level of HMGR activity in a sample obtained from said subject; and (b) comparing said first level to a second level which is an average HMGR activity measured in at least one corresponding control sample obtained from at least one control subject, whereby if said first level is significantly less than said second level then said subject suffers from a dementia; wherein said method is used to diagnose or prognosticate a dementia.

The invention further provides a method of diagnosing or prognosticating a dementia in a subject, said method comprising: (a) obtaining a sample from said subject, wherein said sample comprises ribonucleic acid encoded by an HMGR gene; and (b) determining whether said sample comprises at least one alteration relative to a corresponding control sample obtained from a control subject, wherein said alteration is selected from the group consisting of: (i) an increase in a level of a first ribonucleic acid encoded by an HMGR gene, wherein said first ribonucleic acid has a deletion of exon 13; (ii) an increase in a level of a second ribonucleic encoded by an HMGR gene, wherein said second ribonucleic acid has an insertion of intron M; and (iii) a decrease in a level of a third ribonucleic acid comprising a normal HMGR transcript; wherein the presence of said at least one alteration is used to diagnose or prognosticate a dementia.

In an embodiment, the above-mentioned alteration is determined using reverse transcriptase-polymerase chain reaction (RT-PCR).

The invention further provides a commercial package for the diagnosis and/or the prognostication of a dementia, said commercial package comprising at least one of: (a) means for detecting a polymorphism in an HMGR gene in a sample together with instructions for assessing said polymorphism relative to a corresponding control sample; (b) means for determining a level of HMGR activity in a sample together with instructions for comparing said level with an established standard or a control level measured in at least one corresponding control sample; and (c) means for determining the presence of at least one feature selected from the group consisting of (i) a first HMGR ribonucleic acid having a deletion of exon 13, (ii) a second HMGR ribonucleic acid having an insertion of intron M, (iii) a decrease in a level of a third ribonucleic acid comprising a normal HMGR transcript; and/or an increase in said first and/or second ribonucleic acid relative to said third ribonucleic acid, together with instructions for comparing said feature with a corresponding control feature in a corresponding control sample.

In an embodiment, the above-mentioned sample is a tissue or body fluid of said subject.

In an embodiment, the above-mentioned tissue or body fluid is neural tissue or fluid.

In an embodiment, the above-mentioned tissue or body fluid is selected from saliva, hair, blood, plasma, lymphocytes, cerebrospinal fluid, epithelia and fibroblasts.

In an embodiment, the above-mentioned control subject is a normal age-matched subject.

In an embodiment, the above-mentioned method is used to prognosticate a dementia and wherein the control sample was obtained from the subject at another time.

In an embodiment, the above-mentioned dementia is an Alzheimer disease.

### BRIEF DESCRIPTION OF THE DRAWINGS

FIG. 1: Schematic representation of the postulated cascade of events regulating cholesterol transport during CNS reinnervation. FC: free cholesterol; CE: cholesterol esters; E: apoE.

FIG. 2: Concentration of cholesterol (free and esterified) measured in the brain of Alzheimer disease (n=30) and age-matched control (n=26) subjects.

FIG. 3: Quantitative real time PCR analysis of total HMGR mRNA prevalence in the frontal cortex of control subjects (n=17) and AD subjects (n=23) (mean±S.E.M.). p=0.22 versus control subjects: non-significant.

FIG. 4: Genomic structure of the HMGR gene and its mRNA in the brain. Primers that were used for RT-PCR experiments to delineate exon 13 transcripts are shown.

FIG. 5: HMGR transcript analysis via electrophoretic analysis of PCR products corresponding to each of the three transcripts identified (normal transcript; exon 13 deletion; and intron M insertion).

FIG. 6: HMGR mRNA prevalence in the frontal cortex in Alzheimer disease. Prevalence of the three major forms (normal transcript; exon 13 deletion; and intron M insertion) in autopsy-confirmed AD versus control subjects.

FIG. 7: Correlational analysis of the prevalence of the abnormal HMGR gene transcripts in the brain and the levels of toxic beta amyloid 1-40 in AD and age-matched control subjects.

FIG. 8: DNA Mutation and Polymorphism in the HMGR Gene in Alzheimer Disease

FIG. 9: Sequencing results obtained from one specific Alzheimer disease patient in the vicinity of intron L and exon 13

FIG. 10: PCR amplification of sequence-specific cDNA derived from mRNA extracted from autopsy-confirmed AD and control subjects wherein the upper band is representative of the HMGR mRNA transcript containing intron M.

FIG. 11: Western blot analysis using a specific monoclonal antibody that recognizes a portion of the trans-membrane domain of HMGR as well as the catalytic site of HMGR.

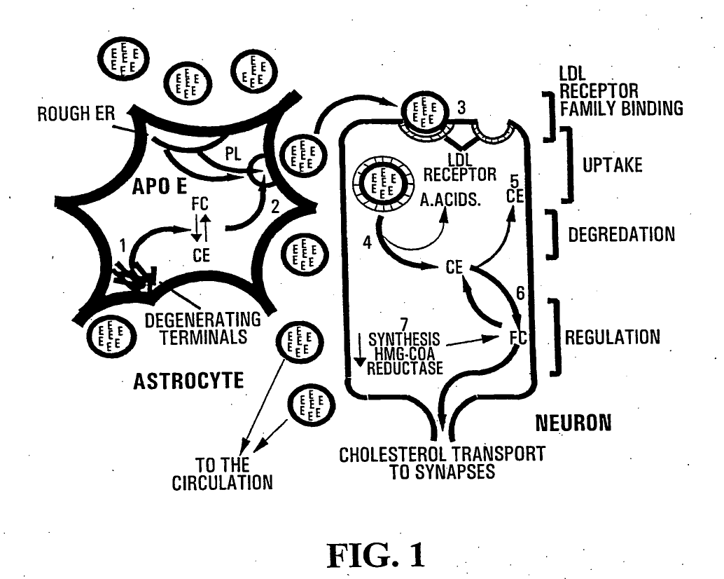

### DETAILED DESCRIPTION OF THE INVENTION

Applicants' findings presented herein reveal that the rate limiting step in the synthesis of cholesterol in the mature brain, the 3-hydroxy-3-methylglutaryl-coenzyme A reductase (HMGR):
a) shows marked reduction in activity in the hippocampal area in AD subjects,
b) displays a significant polymorphic association with sporadic AD (genetic variation localized in intron B (also known as intron 2, eg in hamsters) of the HMGR gene;
c) exhibits the presence of elevated concentrations of two abnormally processed mRNAs in the brain of AD subjects: one mRNA lacks exon 13 whereas the other mRNA contains intron M (the intron between exon 12 and 13).
d) demonstrates brain levels of the toxic beta amyloid peptides 1-40 and 1-42 that markedly (and statistically) correlated with the increased proportion of HMGR mRNA containing the abnormal intron M and lacking exon 13.

None of the age-matched control and parkinsonian subjects examined exhibit this elevated concentration of the two abnormal transcripts. Furthermore, the cerebellum, a brain area that is spared by Alzheimer disease neurodegeneration exhibits concentration of the abnormal transcripts which are within normal range of control subjects, suggesting a selective alteration of the HMGR processing in the portion of the Alzheimer brain targeted by neurodegeneration of the Alzheimer type.

Therefore, applicants submit that the HMGR gene, which acts as a key accessory protein to the apoE/apoE receptor pathway and plays an active role in modulating cholesterol metabolism and beta amyloid production in vitro, is defective in sporadic AD subjects. The loss of HMGR activity is consistent with the reduction of cholesterol synthesis and levels in the AD brain as reported by several independent studies (Gottfries et al., 1996a; Gottfries et al., 1996b; Svennerholm and Gottfries, 1994; Svennerholm et al., 1994; and data presented below) and its indirect effects on amyloid precursor protein metabolism (Bodovitz and Klein, 1996; Mills and Reiner, 1999; Simons et al., 1998).

Applicant has examined candidate genes which are directly involved in the metabolism and/or function of the most important risk factor identified so far, namely the apolipoprotein E4 allele.

Applicants' findings presented herein indicate that the brain expresses three distinct mRNAs for the HMGR in contrast to human liver that normally produces only one form of the enzyme. The presence of two transcripts has been reported previously in embryonic hamster cell line called UT-2 (Aboushadi et al., 2000). The shorter form of the mature mRNA is derived from exon 13 skipping. In the hamster, this shorter form codes for the HMGR localized in peroxisomes. Results obtained in applicants' laboratory using real time quantitative RT-PCR amplification indicate that a very similar processing is occurring in the human brain with a short and long versions of the enzyme which exhibits (or lacks) exon 13 (based on the hamster gene nomenclature). Applicants have confirmed the nature of the different brain transcripts by laser sequencing.

However, in Alzheimer brain, applicants demonstrate that a third rather abnormal, yet prevalent, transcript is being produced in addition to the other two major HMGR mRNAs (see results below). Applicants also demonstrate that the enzymatic activity of the HMGR is reduced by nearly 50% in the brain of AD subjects. The convergence of these experimental findings indicate the abnormal processing of the HMGR transcript (mRNA) in Alzheimer disease, leading, among other things, to a defective protein and reduced tissue enzymatic activity. Furthermore, applicants demonstrate that levels of the toxic beta amyloid 1-40 and 1-42 increase proportionally to the relative concentration of the abnormal HMGR mRNA containing the intron position between exon 12 and 13.

It is well known from genetic studies in the cardiovascular field that full HMGR knockout mice die prematurely in utero, whereas administration of a selective HMGR inhibitor called simvastatin directly into the brain causes lethality at high doses, but loss of white matter at low doses (a characteristic of Alzheimer and vascular dementia). In humans, a near complete absence of cholesterol synthesis due to mutation in dehydrocholesterol reductase gene gives rise to a disease called the Smith-Lemli-Opitz syndrome which is characterized by abnormal myelinization, mental retardation, holoproencephaly and congenital heart disease (Salen et al., 1996).

Applicants have thus identified several events which correlate with Alzheimer disease, including:
(a) a decrease in HMGR activity;
(b) the presence of a polymorphism localized to intron B (also known as intron 2, eg in hamsters) of the HMGR gene;
(c) the presence of two abnormal HMGR mRNAs in the brains of AD subjects, i.e.:
(i)-a first mRNA which lacks exon 13 (designated “transcript #2” in Example 6 below);
(ii) a second mRNA containing intron M (designated “transcript #3” in Example 6 below);
(d) an increase in the level of the two abnormal mRNAs transcript # 2 and transcript #3 relative to levels the normal HMGR mRNA (designated “transcript #1” in Example 6-below; i.e. containing exon 13 and lacking intron M); and
(e) a correlation of the brain levels of beta amyloid peptides 1-40 and 1-42 with the increase in the presence of the two abnormal mRNAs transcript # 2 and transcript #3.

Therefore, the presence of at least one of these events, or in certain embodiments, various combinations of these events, in a subject, is an indication that the subject is suffering from a dementia, such as an Alzheimer disease. Thus, aspects of the present invention are methods for the diagnosis and prognostication of a dementia, such as an Alzheimer disease, via assessing whether a subject exhibits one of the above-mentioned events.

Accordingly, the invention provides a method of diagnosing or prognosticating a dementia, such as an Alzheimer disease, in a subject, said method comprising:
(a) obtaining a sample from said subject, wherein said sample comprises nucleic acid comprising a 3-hydroxy-3-methylglutaryl coenzyme A reductase (HMGR) gene; and
(b) determining whether said nucleic acid comprises a polymorphism relative to a corresponding control sample obtained from a control subject;
   wherein the presence of said polymorphism is used to diagnose or prognosticate a dementia.

In an embodiment, the above-mentioned polymorphism is localized in intron B (also known as intron 2, eg in hamsters) of said HMGR gene. Such a polymorphism may be determined by methods known in the art. In an embodiment, the polymorphism may be determined via restriction fragment length polymorphism analysis (RFLP), for example by amplifying a nucleic acid sequence comprising HMGR intron B (also known as intron 2, eg in hamsters) by polymerase chain reaction (PCR) to obtain a PCR product, digesting the PCR product with a restriction enzyme to obtain a restriction digest product; and examining the length of the restriction digest product. In an embodiment, a suitable restriction enzyme is ScrFI.

The invention further provides a method of diagnosing or prognosticating a dementia, such as an Alzheimer disease, in a subject, the method comprising:
(a) measuring a first level of HMGR activity in a sample obtained from said subject; and
(b) comparing the first level to a second level which is an average of HMGR activity measured in at least one corresponding control sample obtained from at least one control subject, whereby if the first level is significantly less than the second level then said subject suffers from an Alzheimer disease;
   wherein the method is used to diagnose or prognosticate a dementia.

HMGR activity may be assessed or measured using methods known in the art. Various means may be utilized to enable useful assay conditions. Such means may include, but are not limited to suitable buffer solutions, for example, for the control of pH and ionic strength and to provide any necessary components for HMGR activity and stability, temperature control means, and detection means to enable the detection of an HMGR reaction product. In an embodiment, the detection means detects cholesterol. An example of a suitable method for determining HMGR activity is described in Poirier et al, 1993b.

The invention further provides methods of diagnosing or prognosticating a dementia, such as an Alzheimer disease, via the detection of abnormal HMGR mRNA transcripts #2 (lacking exon 13) and/or #3 (including intron M) noted above, and/or via assessing the levels of abnormal HMGR mRNA transcripts #2 and/or #3 relative to the level of the normal HMGR transcript #1 noted above (having exon 13 and lacking intron M)

Accordingly, the invention provides a method of diagnosing or prognosticating a dementia, such as an Alzheimer disease, in a subject, said method comprising:
(a) obtaining a sample from said subject, wherein said sample comprises ribonucleic acid encoded by an HMGR gene; and
(b) determining whether said sample comprises at least one alteration relative to a corresponding control sample obtained from a control subject, wherein said alteration is selected from the group consisting of:
   (i) an increase in a level of a first ribonucleic acid encoded by an HMGR gene, wherein said first ribonucleic acid has a deletion of exon 13;
   (ii) an increase in a level of a second ribonucleic encoded by an HMGR gene, wherein said second ribonucleic acid has an insertion of intron M; and
   (iii) a decrease in a level of a third ribonucleic acid comprising a normal HMGR transcript;
      wherein the presence of said at least one alteration is used to diagnose or prognosticate a dementia.

The above mentioned mRNA transcripts may be detected by various methods known in the art. An example is detection using reverse transcriptase-polymerase chain reaction (RT-PCR), which in an embodiment, is performed in a quantitative manner. Transcripts may for example also be detected by Northern analysis using an appropriate probe(s) (see for example Sambrook et al. (Molecular Cloning: A Laboratory Manual, 2nd Edition, Cold Spring Harbor Laboratory Press [1989], and other laboratory manuals).

The applicants' diagnostic method depends upon a comparison, with control levels, of the level of HMGR enzyme activity in postulated AD subjects, or upon a comparison of the nucleic acids encoding HMGR with those encoding its polymorphisms. The enzyme activity control levels should be established based on analysis of corresponding tissues to the tissue of the AD subject which is being analyzed for HMGR activity. The appropriate tissue for analysis will depend upon the polymorphism as further described elsewhere herein, and more particularly upon whether what is being analyzed is HMGR activity or HMGR encoding nucleic acids. A measured HMGR activity in an appropriate tissue sample obtained from an AD subject which exhibits a statistically significant reduction over the corresponding average level of HMGR activity in corresponding tissue of controls is a clear indication of dementia, particularly AD. Applicants suggest that any reduction of brain HMGR activity greater than 30% equivalent to one standard deviation) of control range values should be considered etiologically associated to common Alzheimer disease.

In embodiments, the above-mentioned sample is a tissue or body fluid of said subject, in a further embodiment, a neural tissue or fluid. Suitable tissue or body fluids include but are not limited to saliva, hair, blood, plasma, lymphocytes, cerebrospinal fluid, epithelia, neural cells and fibroblasts.

In an embodiment, the above mentioned control subject is a normal age-matched subject. In a further embodiment, the above mentioned methods are used for prognostication and the control sample is obtained from the subject at another time, in an embodiment, at an earlier time.

In embodiments, the above mentioned diagnostic and prognostic methods may be utilized independently or in further embodiments in various combinations. For example, the diagnostic and prognostic methods which detect polymorphisms of HMGR may be used to identify subjects at risk of developing a dementia, particularly an Alzheimer disease, and thereby permit appropriate precautionary or preventative treatment to be undertaken. Examples of such treatments include administering to such “at risk” subjects HMGR inhibitors such as statins.

The invention further relates to commercial packages or kits for carrying out the therapeutic, prophylactic, diagnostic and screening methods noted above, comprising the appropriate above-mentioned reagents together with instructions for methods of diagnosis and/or prognostication of a dementia, such as an Alzheimer disease.

Accordingly, the invention further provides a commercial package for the diagnosis and/or the prognostication of a dementia, such as an Alzheimer disease, said commercial package comprising at least one of:
(a) means for detecting a polymorphism in an HMGR gene in a sample together with instructions for assessing said polymorphism relative to a corresponding control sample;
(b) means for determining a level of HMGR activity in a sample together with instructions for comparing said level with an established standard or a control level measured in a corresponding control sample; and
(c) means for determining the presence of at least one feature selected from the group consisting of (i) a first HMGR ribonucleic acid having a deletion of exon 13, (ii) a second HMGR ribonucleic acid having an insertion of intron M, (iii) a decrease in a level of a third ribonucleic acid comprising a normal HMGR transcript; and/or an increase in said first and/or second ribonucleic acid relative to said third ribonucleic acid,
together with instructions for comparing said feature with a control feature in a corresponding control sample.

The commercial package may be used for the analysis of samples as discussed above.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

Throughout this application, various references are referred to to describe more fully the state of the art to which this invention pertains. The disclosures of these references are hereby incorporated by reference into the present disclosure.

### EXAMPLES

### Example 1

### Cholesterol Levels in the Brain of Alzheimer Disease Significant Reduction of Both Free Cholesterol and Cholesterol Esters (Stored Cholesterol) in the Temporal Cortex of Autopsy-Confirmed AD Subjects

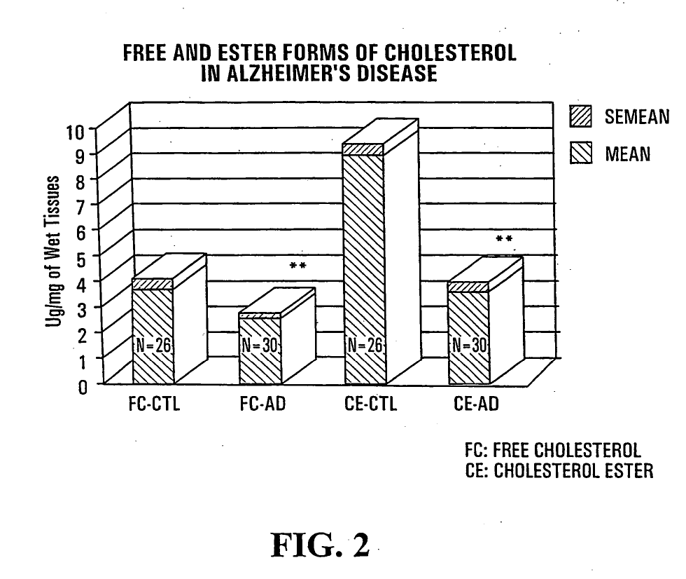

FIG. 2 illustrates the concentration of cholesterol (free and esterified) measured in the brain of Alzheimer disease (n=30) and age-matched control (n=26) subjects. A significant reduction of steady stage cholesterol levels could be observed in AD subjects. The reduction affects both the free and esterified forms of cholesterol. These results are consistent with previous literature on the subject (Gottfries et al., 1996a; Gottfries et al., 1996b; Svennerholm and Gottfries, 1994; Svennerholm et al., 1994).

TABLE 1

| | HMGR-CoA Reductase activity in Alzheimer's | | | |
|---|---|---|---|---|
| | Disease and Control Subjects | | | |
| | Control | Alzheimer | Alzheimer | Alzheimer |
| | 3/3 | 3/3 | 4/3 | 4/4 |
| Activity | 46.1 | 30.04 | 28.74 | 23.57 |
| Mean | | | | |
| (units/mg/min) | | | | |
| S.E.M. | 13.12 | 10.12 | 6.49 | 5.54 |
| N= | 10 | 9 | 12 | 12 |
| Statistics | | p < 0.046 | p < 0.039 | p < 0.010 |
| (T-Test) | | | | |

### Example 2

### HMGR Enzymatic Activity: Rate Limiting Step in Cholesterol Synthesis

Analysis of HMG-CoA activity in temporal cortices revealed a significant reduction in AD versus age-matched control subjects (Table 1). The reduction in enzymatic activity (which reaches about 50% in the apoE4/4 genotype) is not specific to apoE4 allele carriers. It affects nearly all patients suffering from sporadic AD.

Prevalence analysis of total HMGR mRNA (all forms) indicates, on the other hand, a slight (non-significant) reduction of the total HMGR transcript prevalence in AD subjects (FIG. 2). The methodology pertaining to the HMGR-CoA reductase activity and mRNA prevalence in the brain have been described previously (Poirier et al., 1993).

Statistical analysis using correlational analysis (SPSS Linear Regression: activity vs gene dose) did not reveal any significant E4 allele-dose effect on the activity of the HMGR. Sex, gender and post-mortem delays do not explain the differences between AD and age-matched control subjects (SPSS Covariance Analysis: negative).

These results indicate the presence of an anomaly in the HMGR-CoA reductase metabolism which minimally affects global gene expression levels but affects enzymatic activity in the brain of AD subjects. This reduction in cholesterol synthesis in the brain is consistent with the observed reductions of cholesterol (free and esterified) in the brain of AD subjects.

### Example 3

### HMGR mRNA Prevalence in Temporal Cortex of AD Subjects

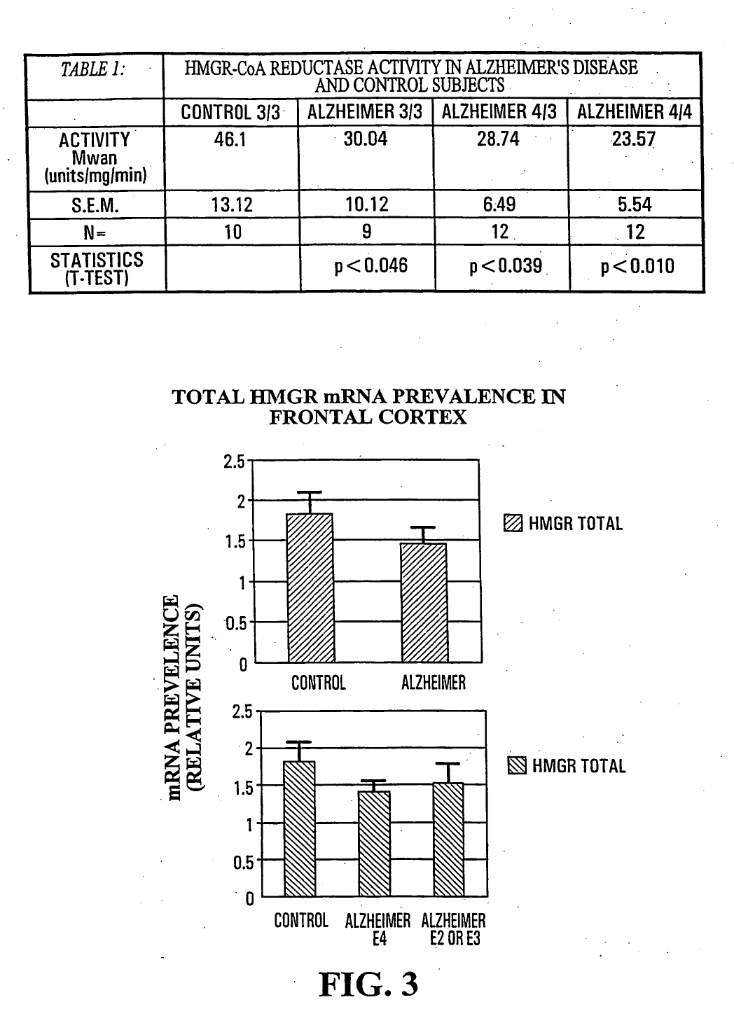

FIG. 3 illustrates results obtained from quantitative real time PCR analysis of total HMGR mRNA prevalence in the frontal cortex of 23 AD subjects and 17 age-matched control subjects. E4 allele carriers as well as non-E4 subjects exhibit similar (lower) levels of total HMGR mRNAs. The frontal cortex is an area of marked neuronal cell damage in Alzheimer disease.

The reduction in both cholesterol synthesis (Table 1) and mRNA prevalence of HMGR is very consistent with scientific reports that claim that cholesterol levels in the brain of Alzheimer disease are markedly decreased when compared to control subjects (Nitsch et al., 1992, Svennerholm et al., 1994).

### Example 4

### Analysis of a Polymorphic Marker in the HMGR Gene in Sporadic Alzheimer Disease

To address the issue of possible polymorphisms/mutations in the HMGR gene that would affect mRNA processing or enzyme activity, applicants have used a gene/disease association approach to assess the role of a genetic contribution to sporadic Alzheimer disease.

The genomic sequence of the human HMGR gene was made available only recently in public databases. The presence of the common polymorphism localized in intron B (also known as intron 2, eg in hamsters) of the HMGR gene has been reported (Leitersdorf et al., 1990). It should be noted that the gene locus of the HMGR gene on chromosome 5 is only a few centimorgans away from one of the so-called “hot spots” identified by Dr. Allison Goates and her group in a genome wide scan performed in late onset familial cases (Kehoe et al., 1999). This particular region of chromosome 5 is believed to contain a susceptibility gene or a disease-causing gene for familial late onset AD.

The assay consists of a PCR amplification of the intron B (also known as intron 2, eg in hamsters) area followed by a digestion with the restriction enzyme ScrFI. The digestion leads to the formation of 2 bands (heterozygotes), one of which is 120 bp long and the other of which is 165 bp long (Leitersdorf et al., 1990). Table 2 summarizes the frequency distribution results obtained in applicants' pilot study with 84 autopsy-confirmed control and 64 AD subjects. Groups were matched for gender, age and ethnicity. The association between allele L and sporadic AD was estimated by chi-square analysis and found to be statistically significant (SPSS statistical program). The odds ratio is 1.8 with a 95% confidence interval of 1.1 to 3.1. The autopsy-confirmed elderly control subjects do not differ from the published population prevalence. These results clearly indicate the L allele is linked to sporadic AD in this cohort of autopsy-confirmed subjects.

TABLE 2

Frequency Distribution

| Subjects | Allele D | Allele L | Significance |
|---|---|---|---|
| North American | 0.45 | 0.55 | |
| Population | | | |
| Autopsy- | 0.48 | 0.52 | NS vs |
| Confirmed | | | Population |
| Controls | | | |
| Autopsy- | 0.34 | 0.66* | p < 0.01 |
| Confirmed | | | (vs controls) |
| Alzheimers | | | |

### Example 5

### Abnormal Processing of the HMGR mRNA in Alzheimer Disease

After a careful scan of the literature for possible HMGR mutations in humans, applicants have come to the conclusion that any significant modification of the HMGR gene through insertion or deletion may be so detrimental that no humans' can survive with such a major defect. Conversely, it could also indicate the presence of an effective compensatory mechanism that dampens a more deleterious effect. HMGR mRNA anomalies in UT2 cells which are mutant clones of Chinese Hamster Ovary cells have been reported (Reynolds, et al., 1985). UT2 cells are completely deficient in 97 Kd endoplasmic reticulum HMGR protein involved in cholesterol synthesis. The defect arises from a mutation in the splice site causing exon 11 skipping (an mRNA without exon 11). The peroxisome HMGR protein and its mRNA are apparently intact in this cell type. The mutation causes aberrant splice messages that are easily identifiable by PCR.

Applicants examine herein the mRNA structure of HMGR between exon 11 and 15 in AD and control subjects using the published sequence of the cDNA available in GENEBANK (Genebank accession number: L00166 and AH001819). This particular region of the mRNA contains the catalytic site of the enzyme.

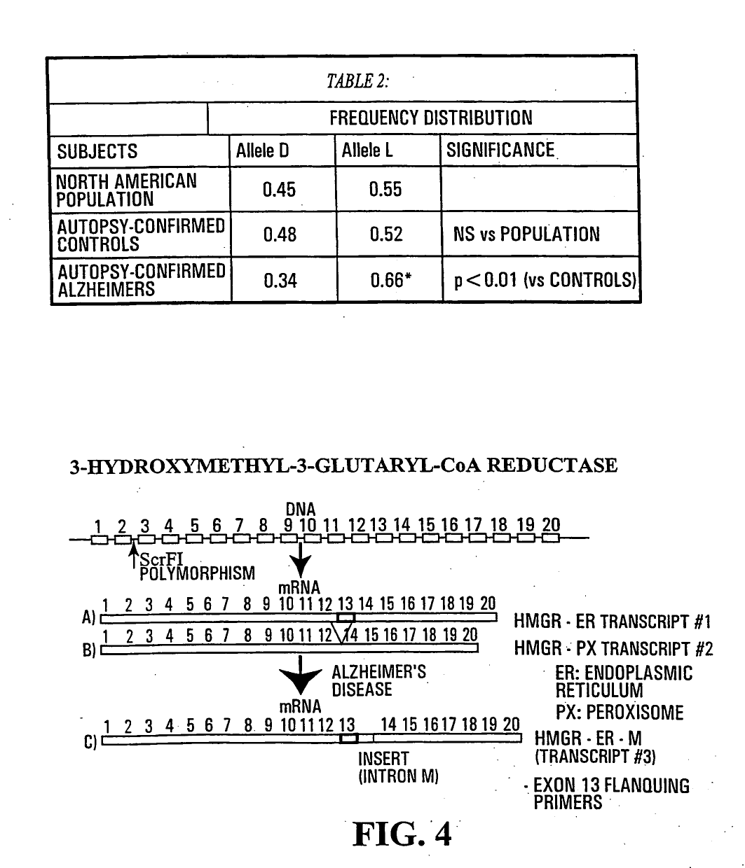

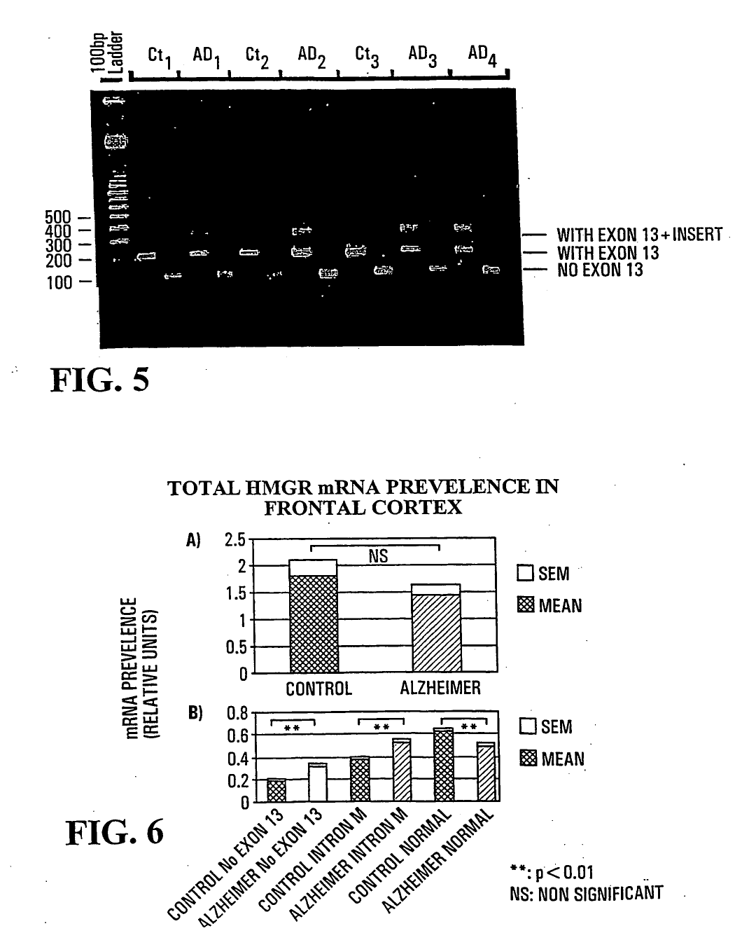

The hamster genomic map was used as a reference to establish a working map of the human genomic structure (Genebank L00166 and AH001819). Applicants thus considered that exon skipping, insertion or deletion in one of the HMGR transcripts could explain the partial loss of activity observed in the AD brain without affecting the normal sterol-mediated up regulation of gene expression. Indeed, applicants have discovered that the human brain expresses two major mRNAs for the HMGR: one without exon 13 and one with exon 13 (FIG. 4). However, both control and AD subjects express the shorter form of the HMGR (the peroxisomal form) and long version (ER) of the transcript as shown in FIG. 3. Applicants have developed specific DNA primers to amplify the cDNA that contains the exon 13 insert, and another set of primers that amplifies only the transcript that lacks exon 13 (FIGS. 4 and 5). Applicants have used a modified version of the method described by Powell and Kroon for HMGR mRNA quantification (Powell and Kroon, 1992). Amplification of the transcript that contains exon 13 also gives rise to an additional PCR product which is much longer that the anticipated DNA (FIG. 4, see exon 13+insert in the HMGR-AD transcript and FIG. 5, top band in the exon 13 lane of the three AD subjects).

Furthermore, this longer band was found to be highly prevalent (visible) in the brain of AD subjects when compared to control age-matched subjects. FIG. 5 illustrates the corresponding band profile obtained after RT-PCR and gel electrophoresis of the mRNA containing (or not) exon 13. Applicants have repeated these analyses in a small group of parkinsonians to examine the HMGR transcript profile in another neurodegenerative disease characterized by glial cell proliferation.

Results were identical to the age-matched normal control subjects. Applicants repeated the analysis with 13 additional AD, 23 control and 10 parkinsonian subjects. Only AD subjects exhibit the intense band corresponding to exon 13+insert. Furthermore, applicants have examined the HMGR transcript profile in the human glioblastoma cell line HTB-14 (ATCC collection: apoE genotype 0.3/3) and found the usual short and a long bands, without the abnormal “exon 13+insert” mRNA (not shown). The last two experiments rule out the possibility that the abnormal exon 13 band could be the result of glial cell proliferation (especially astrocyte enrichment). The unknown 400 bp band (exon 13+insert) initially appeared to be the result of an abnormal processing of the transcript in the area of exon 13 of the HMGR gene. Exons 12, 13 and 14 contain key acidic residues required for the functional catalytic activity of the HMGR (Frimpong and Rodwell, 1994; Wang et al., 1990). The longer transcript carrying the exon 13 is the mRNA responsible for the synthesis of the ER-bound HMGR; the form of HMGR that regulates cholesterol synthesis.

### Example 6

### Sequencing of the Different HMGR Transcripts

In a series of follow-up experiments, applicants have used sequence specific primers to amplify the mRNA coding for the different HMGR transcripts and laser sequencing to determine the nucleic acid sequence of each of the transcripts. Applicants confirmed that the two major species of HMGR mRNAs in the human brain contain either exon 2 to 20, or exon 2 to 20 without exon 13 (exon 13 skipping).

Applicants also demonstrated that there are two types of HMGR transcripts. One transcript contains exon 2 to 20 as expected whereas a second transcript contains exons 2-13, intron M, exons 14 to 20. The second transcript which contains the entire intron M (which is localized between exon 13 and 14) appears to be the result of an abnormal splicing of the HMGR pro-transcript.

Analyses of the amplified transcripts (with and without intron M) clearly indicate an increased prevalence of the HMGR transcript containing the intron M and a marked reduction of the “normal”. HMGR transcript containing exon 2 to 20 when compared to age-matched control subjects. These findings lead applicants to design a quantitative real time reverse transcript PCR protocol aimed at determining the actual prevalence of the three major HMGR transcripts:
Transcript #1): exons 0.2 to 20
Transcript #2): exons 2 to 20, without exon 13
Transcript #3): exons 2 to 20, plus intron M (between exons 13 and 14)

Primers were designed to amplify by real time quantitative RT-PCR, pieces of cDNA that corresponded to Transcripts #1, #2 and #3. Another pair of primers was designed to quantify the total mRNA prevalence of the HMGR in the brain, irrespective of the exon 13 modifications (FIG. 6A). This pair of primers amplifies a portion of exon 14. The RT-PCR fragments corresponding to Trancripts #1, #2 and #3 are of similar length, with similar CG composition so that the proportion of each transcript species is determined in relation to the total HMGR mRNA prevalence. Furthermore, beta actin levels were estimated in each samples and used as an internal, non changing, standard.

The analysis was performed in autopsy confirmed. Alzheimer disease, Parkinson disease and control age-matched subjects. Three different brain areas were used: the frontal and temporal cortices which are affected by AD's neuropathology and, the cerebellum which is spared by the disease process. FIG. 6B illustrates the relative concentration of Transcript #2 (HMGR without exon 13) in the frontal cortex, in AD versus control subjects. There is a statistically significant increase (p=0.003) of this abnormal transcript in the cortical areas of AD subjects. Similar analyses were performed in the brains of Parkinson disease (PD) subjects- and applicants found that the prevalence of Transcript #2 in PD is very similar to that of age-matched control subjects (not shown); this suggests that the increase found in AD brain is not simply the result of neurodegenerative losses but of some more fundamental changes in the etiophathology of Alzheimer disease per se. The cerebellum reveals levels of Transcripts #1, #2 and #3 in AD which are similar to control subjects.

Therefore, the AD brain is characterized by an increased prevalence of HMGR transcript without exon 13 (exon skipping) in brain areas damaged by the disease process.

Applicants also examined the relative proportion of Transcripts #1 and #3 as they both represent the full length HMGR mRNA (with and without intron M). Applicants found that the proportion of transcripts containing the abnormal intron M increases markedly in the brain of AD subjects (p=0.002) at the expense of the “normal” HMGR mRNA (p<0.01) which is used for the synthesis of cholesterol. These results explain why the activity of the HMGR protein decreases in the brain of Alzheimer disease subjects (shown in Table 1) as the production of the two abnormal transcripts either containing intron M or lacking exon 13 is done at the expense of the synthesis of the normal form transcript for the HMGR mRNA. Furthermore, it is interesting to note that both forms of the abnormal HMGR mRNA are produced by the abnormal splicing of the pro-RNA (exon skipping or intron retention).

Therefore, the presence of the two abnormal transcripts #2 and #3, particularly the transcript #3 (with the intron M), is consistent with a gain of toxic function in the AD brain whereas the (concomitant) loss of the normal HMGR transcript #1 coincides with the reduction of enzymatic activity observed in the AD brain.

### Example 7

### Beta Amyloid, Alzheimer Disease Pathology and MGR mRNA Processing

Review of the recent literature indicates evidence of a tight biochemical association between cholesterol production and levels in the brain of Alzheimer disease patients and the production of toxic beta amyloid peptides (for a review, see Poirier, 2000). The beta amyloid peptide is known to polymerize in the brain of Alzheimer subjects and to cause the accumulation of the so-called toxic beta amyloid deposits. Because of the well known contribution of beta amyloid to the pathophysiology of Alzheimer disease, applicants examined the effect of the alteration in HMGR transcript prevalence in relation to the production of the two major forms of beta amyloid in the brain of AD subjects: the beta amyloid 1-40 and the beta amyloid 1-42.

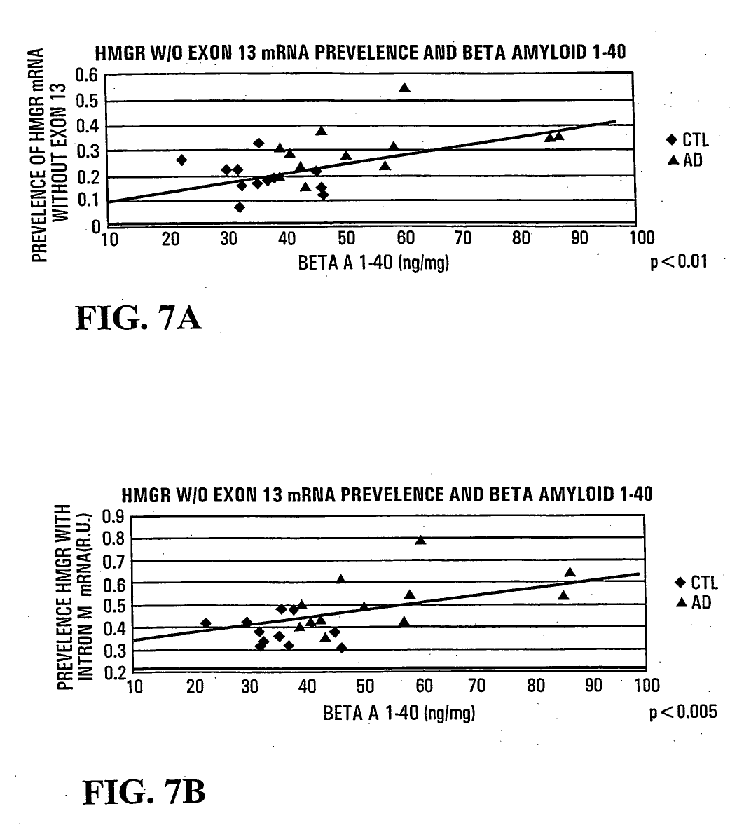

FIG. 7 illustrates the correlational analysis that was performed using applicants' AD and control patient populations. Results clearly indicate that the increased production (and levels) of beta amyloid in the brain of AD subjects is tightly associated with the increased prevalence of the abnormal transcripts #2 or #3 (p<0.005). In contrast, there is no association between the “normal HMGR transcript” prevalence and the levels of beta amyloid peptides in the brain of applicants' subjects (not shown). The marked dichotomy between the control subjects (circles) and Alzheimer disease patients (triangles) is also noted.

Therefore, AD subjects expressing the highest incidence of abnormal transcripts # 2 and #3 also exhibit the highest levels of beta amyloid 1-40 in the brain.

### Example 8

### Experimental Methods

Sequencing of the HMG-CoA reductase gene and mRNAs using brain tissues from autopsy confirmed cases of Alzheimer disease and control subjects:

Brain Tissues: The studies described above examined the nucleic acid sequence of the HMGR gene in genetically characterized AD cases (apoE4 and non-E4), neurological controls [Parkinson (PD) disease] as well as intact age-matched control subjects. Frozen tissues from the frontal cortices were obtained from the Douglas Hospital Brain Bank. One hundred and fifteen intact controls, 9 idiopathic PD subjects with no AD pathology, and 153 AD subjects have been genotyped in the bank and the DNA is available for genetic analyses. Cases are without formal pattern of family inheritance (sporadic subjects). The pathological criteria in use at the Douglas Hospital brain bank have been described before (Poirier et al., 1995).

Methodology: The sequencing methodology used in this project is well documented and adapted from a strategy that applicants have used in the past for the LDL receptor sequencing (Arguin et al., 1997). Two automated ALF laser sequencers from Pharmacia Biotech were used to run the sequencing reactions and to analyse the sequences.

Statistical analyses were performed by SPSS (Statistical Package for Social Science software, version 7) using multivariate logistic regressions and correlation analyses adjusted for age and gender.

Analysis of the polymorphic association between HMGR intron B (also known as intron 2, eg in hamsters) and Alzheimer disease:

Assessment of the prevalence of the different polymorphisms in intron B (also known as intron 2, eg in hamsters) of the HMGR gene was performed as described by Leitersdorf, et al. (1990) using PCR amplification followed by restriction enzyme digestion (with ScfRI restriction enzyme). The prevalence of each allele (D and L) was determined for 64 Alzheimer disease subjects and 84 age-matched control subjects with no known neurological diseases. Aged subjects exhibited prevalence of D and L alleles similar to population studies published by Leitersdorf et al. (1990). In contrast, the AD cohort showed a statistically significant alteration of the D and L allele prevalence when compared to control cohorts.

Analysis of HMGR mRNA prevalence of the different transcripts and assessment of the HMGR activity in different areas of the CNS in AD and age-matched control subjects:

Analysis of the HMGR mRNA prevalence was performed using quantitative real time RT-PCR with sequence-specific primers that delineate exon 13 according to a modification of the Powell et al. (1992) method. The following primers:

| | |
|---|---|
| Primer 6PS: | CTCTTGCTTGGTGGAGGTG |
| Primer 18AS: | TGACTCTGCAGAAGTGAAAGCCTGGC |
| Primer 6GS: | CTCCTTGGTGATGGGAGCT |
| Primer 6GAS: | GTCCTTGCAGATGGGATGAC |

were used to selectively amplify segments of reverse transcripted RNA that contained nucleic sequences with and without exon 13. This technique allowed the segregation of mRNAs with and without exon 13. The real time quantitative RT-PCR technology allows us to monitor simultaneously different isoforms of a given mRNA and, to examine structural instability of the different isoforms by means of temperature dissociation profile. The dissociation profile allows the amplification of the normal; and abnormal transcripts, and to independently assess the relative amount of each transcript in a given brain area. In these experiments, actin was used as positive control transcripts.

Enzymatic activity for HMGR in the brain was assessed as described in Poirier et al., 1993b.

### Example 9

### HMGR Genetic Variants

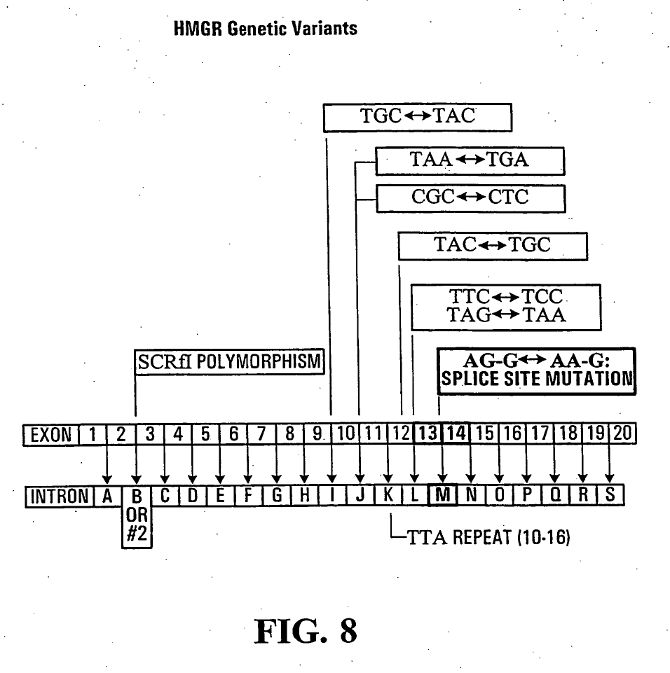

FIG. 8 illustrates several of the polymorphisms found in applicants' analysis of the HMGR DNA of various Alzheimer disease subjects.

The DNA of one of applicants' AD subjects (known as subject 993) is characterized by the presence of a mutation at the frontier of intron L and exon 13, in the so-called splice site junction sequence. This mutation replaces a key guanine (G) by an adenosine (A) in the consensus splice site between intron L and exon 13. This anomaly (which is heterozygous, i.e. present in only one allele) in the particular AD subject causes mis-processing of the HMGR mRNA and the retention of exon M in the mature transcript. This type of intron retention due to splice site mutations has been described for other diseases, but never before for Alzheimer disease. Since this anomaly involves a DNA mutation, and thus can be identified by a DNA test, this anomaly is readily detectable, not only in the brain which is affected by Alzheimer disease, but also in other tissues which do not exhibit symptoms of the disease, such as saliva, blood and hair.

The other polymorphisms described in FIG. 8 (as identified by laser sequencing) in the introns I, J, K are also examples of genetic modifications in the HMGR transcript.

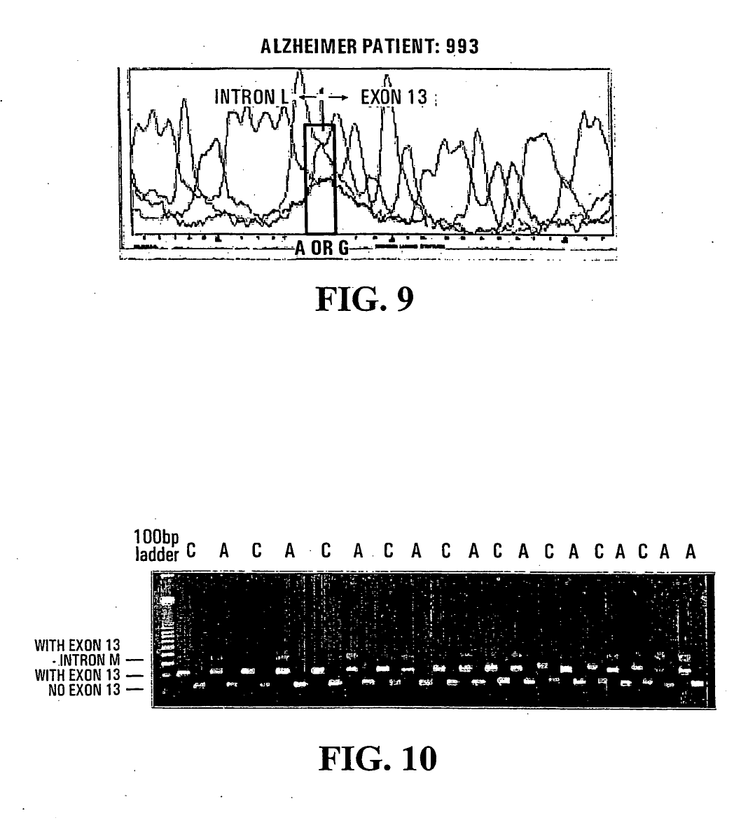

FIG. 9 is a printout of the sequencing results obtained from Alzheimer disease patient 993 in the vicinity of intron L and exon 13. A mutation at position 2 of the splice site junction (A versus G) is known to cause intron retention (and some times also exon skipping) in several independent genes in humans and animals. Interestingly, this subject is homozygous for allele L in intron B (also known as intron 2, eg in hamsters), which is the allele applicants have found to be strongly associated to common Alzheimer disease. It is thus no coincidence that both genetic anomalies are strongly linked to AD since they affect HMGR processing in a manner consistent with the presence of abnormal protein and loss of HMGR activity.

FIG. 9 illustrates the sequencing output of the DNA sequencer demonstrating the presence of the two alleles (A or G) in the DNA of subject 993 suffering from Alzheimer disease. This finding provides an explanation (at least for this particular patient) of why Alzheimer disease subjects demonstrate abnormal splicing of the HMGR gene, causing intron M retention in all AD subjects examined so far. Because the mutation was found at the level of the DNA, genetic tests designed to identify mutations like these in the HMGR gene could be performed in any type of human tissue including blood, biopsy or hair.

Applicants have expanded on their original observation of the presence of the abnormal intron M in the mRNA of the HMGR. In FIG. 10, applicants illustrate amplification of the Exon 13-Intron M-Exon 14 in autopsy-confirmed AD (n=10) and Control (n=9) brains. While all of the AD brains exhibit the abnormal form of the transcript (containing the intron M), only one age-matched control (the last one on the right) exhibits the abnormal band. Since the sample was obtained from an autopsy subject, it is not possible to determine if the control was, in fact, a non-symptomatic, possible future Alzheimer subject.

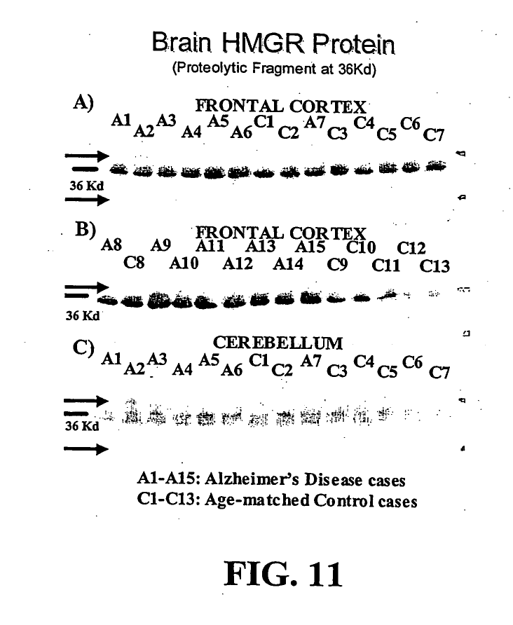

FIG. 11 examines the consequence of the presence of the abnormal transcript on the HMGR protein profile by Western blot analysis using a specific monoclonal antibody that recognizes a portion of the trans-membrane domain of the enzyme as well as the catalytic site of the enzyme. Since the activity of the enzyme was shown to be markedly reduced in the Alzheimer brains, applicant expected (and found) the presence of abnormal bands in the western blot analysis. A band can be observed on top of the expected HMGR doublet at 36 Kd and in some patients, another abnormal band can be oberved below the 36 Kd reference HMGR bands. The high molecular weight band corresponds to the approximate molecular weight of a truncated form of the HMGR protein that retains the intron M which exhibits a stop codon in the first few nucleic acids of the intron. That truncated form of the HMGR protein is not only expressed in areas of the brain where the disease is most severe but also in the cerebellum, an area relatively free of pathology. This observation of a widespread expression of a truncated form of the HMGR in the brain of AD brain is very consistent with a genetic (widespread) defect causing abnormal splicing of the HMGR transcript and expression of a defective protein. This mutation in the DNA can be observed in samples taken from tissues throughout the subject's body, for example in the brain, cerebellum, liver, hair, saliva, hair and blood.

Thus, this mutation, in combination with the modifications in intron B (also known as intron 2, eg in hamsters) described earlier, are clear examples of mutation/genetic anomalies that are detected by genetic testing or screening of DNAs and linked to Alzheimer disease. Furthermore, the abnormal splice site mutation discovered at the junction of intron L and exon 13 also represents an example of a mutation that causes intron retention which in turn affects mRNA splicing, causing abnormal protein production and finally, loss of enzymatic activity and function. Since exons 12, 13 and 14 are the regions which give rise to the catalytic site of the enzyme, other mutations/polymorphisms in these regions of the HMGR gene will be of particular utility in applicants' diagnostic method.

### REFERENCES

Aboushadi, N., Shackelford, J. E., Jessani, N., Gentile, A., and Krisans, S. K. (2000). Characterization of peroxisomal 3-hydroxy-3-methylglutaryl coenzyme A reductase in UT2 cells: sterol biosynthesis, phosphorylation, degradation, and statin inhibition. Biochemistry 39, 237-247.
Arendt, T., Schindler, C., Bruckner, M. K., Eschrich, K., Bigl, V., Zedlick, D., and Marcova, L. (1997). Plastic neuronal remodeling is impaired in patients with Alzheimer's disease carrying apolipoprotein epsilon 4 allele. JNS 17, 516-529.
Arguin, C., Theroux, L., Danik, M., and Poirier, J. (1997). Screening for mutations in the low-density lipoprotein receptor gene in Alzheimer's Disease patients. Soc. Neurosci. Abstr. 23, 82.
Bales, K. R. and et al (1997). Lack of apolipoprotein E dramatically reduces amyloid □-peptide deposition. Nature Medicine 17, 263-264.
Baum, L., Chen, L., Masliah, E., Chan, Y. S., Ng, H. K., and Pang, C. P. (1999). Lipoprotein lipase mutations and Alzheimer's disease. Am. J. Med. Genet. 88, 136-139.
Beffert, U., Arguin, C., and Poirier, J. (1999a). The polymorphism in exon 3 of the low density lipoprotein receptor-related protein gene is weakly associated with Alzheimer's disease. Neurosci. Lett. 259, 29-32.
Beffert, U., Aumont, N., Dea, D., Davignon, J., and Poirier, J. (1996). Apolipoprotein E uptake is increased by beta-amyloid peptides and reduced by blockade of the LDL receptor. Neurodegenerative Diseases 103-108.
Beffert, U., Aumont, N., Dea, D., Lussier-Cacan, S., Davignon, J., and Poirier, J. (1998b). Beta-amyloid peptides increase the binding and internalization of apolipoprotein E to hippocampal neurons. J. Neurochem. 70, 1458-1466. Beffert, U., Aumont, N., Dea, D., Lussier-Cacan, S., Davignon, J., and Poirier, J. (1999b). Apolipoprotein E isoform-specific reduction of extracellular amyloid in neuronal cultures. Molecular Brain Research 68, 181-185.
Beffert, U., Cohn, J. S., Petit-Turcotte, C., Tremblay, M., Aumont, N., Ramassamy, C., Davignon, J., and Poirier, J. (1999c). Apolipoprotein E and beta-amyloid levels in the hippocampus and frontal cortex of Alzheimer's disease subjects are disease-related and apolipoprotein E genotype dependant. Brain Research 843, 87-94.
Beffert, U., Danik, M., Krzywkowski, P., Ramassamy, C., Berrada, F., and Poirier, J. (1998a). The neurobiology of apolipoproteins and their receptors in the CNS and Alzheimer's disease. Brain Res. Brain Res. Rev. 27, 119-142.
Beffert, U. and Poirier, J. (1998). ApoE associated with lipid has a reduced capacity to inhibit beta-amyloid fibril formation. Neuroreport 9, 3321-3323.
Bertrand, P., Poirier, J., Oda, T., Finch, C. E., and Pasinetti, G. M. (1995). Association of apolipoprotein E genotype with brain levels of apolipoprotein E and apolipoprotein J (clusterin) in Alzheimer disease. Brain Research Molecular Brain Research. 33, 174-178.
Bodovitz, S. and Klein, W. L. (1996). Cholesterol modulates alpha-secretase cleavage of amyloid precursor protein. J. Biol. Chem. 271, 4436-4440.
Boyles, J. K., Notterpek, L. M., and Anderson, L. J. (1990). Accumulation of apolipoproteins in the regenerating and remyelinating mammalian peripheral nerve. Identification of apolipoprotein D, apolipoprotein A-IV, apolipoprotein E, and apolipoprotein A-I. J. Biol. Chem. 265, 17805-17815.
Brandi, M. L., Becherini, L., Gennari, L., Racchi, M., Bianchetti, A., Nacmias, B., Sorbi, S., Mecocci, P., Senin, U., and Govoni, S. (1999). Association of the estrogen receptor alpha gene polymorphisms with sporadic Alzheimer's disease. Biochem. Biophys. Res. Commun. 265, 335-338.
Brown, M. S., Dana, S. E., and Goldstein, J. L. (1973). Regulation of 3-hydroxy-3-methylglutaryl coenzyme A reductase activity in human fibroblasts by lipoproteins. Proc Natl Acad Sci U S A 70, 2162-2166.
Chartier-Harlin, M. C., Crawford, F., Houlden, H., Warren, A., Hughes, D., Fidani, L., Goate, A., Rossor, M., Roques, P., and Hardy, J. (1991). Early-onset Alzheimer's disease caused by mutations at codon 717 of the beta-amyloid precursor protein gene. Nature 353, 844-846.
Chin, D. J., Gil, G., Russell, D. W., Liscum, L., Luskey, K. L., Basu, S. K., Okayama, H., Berg, P., Goldstein, J. L., and Brown, M. S. (1984). Nucleotide sequence of 3-hydroxy-3-methyl-glutaryl coenzyme A reductase, a glycoprotein of endoplasmic reticulum. Nature 308, 613-617.
Choi, J. W. and Choi, H. S. (2000). The regulatory effects of thyroid hormone on the activity of 3-hydroxy-3-methylglutaryl-coenzyme A reductase. Endocr. Res. 26, 1-21.
Corder, E. H., Saunders, A. M., Strittmatter, W. J., Schmechel, D. E., Gaskell, P. C., Small, G. W., Roses, A. D., Haines, J. L., and Pericak-Vance, M. A. (1993). Gene dose of apolipoprotein E type 4 allele and the risk of Alzheimer's disease in late on set families [see comments]. Science 261, 921-923.
Cronin, S. R., Khoury, A., Ferry, D. K., and Hampton, R. Y. (2000). Regulation of HMG-CoA reductase degradation requires the P-type ATPase Cod1p/Spf1p. J. Cell Biol. 148, 915-924.
Farrer, L. A., Abraham, C. R., Haines, J. L., Rogaeva, E. A., Song, Y., McGraw, W. T., Brindle, N., Premkumar, S., Scott, W. K., Yamaoka, L. H., Saunders, A. M., Roses, A. D., Auerbach, S. A., Sorbi, S., Duara, R., Pericak-Vance, M. A., and George-Hyslop, P. H. (1998). Association between bleomycin hydrolase and Alzheimer's disease in Caucasians. Ann. Neurol. 44, 808-811.

Farrer, L. A., Cupples, A., Haines, J. L., Hyman, B. T., Kukull, W. A., Mayeux, R., Myers, R. H., Pericak-Vance, M. A., Risch, N. J., and van Duijn, C. M. (1997). Effects of age, sex and ethnicity on the association between apolipoprotein E genotype and Alzheimer disease. A Meta analysis. JAMA 278, 1349-1356.
Frimpong, K. and Rodwell, V. W. (1994). The active site of hamster 3-hydroxy-3-methylglutaryl-CoA reductase resides at the subunit interface and incorporates catalytically essential acidic residues from separate polypeptides. J. Biol. Chem. 269, 1217-1221.
Gardner, R. G. and Hampton, R. Y. (1999). A highly conserved signal controls degradation of 3-hydroxy-3-methylglutaryl-coenzyme A (HMG-CoA) reductase in eukaryotes. J. Biol. Chem. 274, 31671-31678.
Gottfries, C. G., Jungbjer, B., Karlsson, I., and Svennerholm, L. (1996a). Reductions in membrane proteins and lipids in basal ganglia of classic Alzheimer disease patients. Alzheimer Dis. Assoc. Disord. 10, 77-81.
Gottfries, C. G., Karlsson, I., and Svennerholm, L. (1996b). Membrane components separate early-onset Alzheimer's disease from senile dementia of the Alzheimer type. Int. Psychogeriatr. 8, 365-372.
Hardy, J. (1994). ApoE, amyloid, and Alzheimer's disease [letter]. Science 263, 454-455.
Holtzman, D. M., Bales, K. R., Wu, S., Bhat, P., Parsadanian, M., Fagan, A. M., Chang, L. K., Sun, Y., and Paul, S. M. (1999). Expression of human apolipoprotein E reduces amyloid-beta deposition in a mouse model of Alzheimer's disease. J. Clin. Invest 103, R15-R21.
Howland, D. S., Trusko, S. P., Savage, M. J., Reaume, A. G., Lang, D. M., Hirsch, J. D., Maeda, N., Siman, R., Greenberg, B. D., Scott, R. W., and Flood, D. G. (1998). Modulation of secreted β-amyloid precursor protein and amyloid □-peptide in brain by cholesterol. J. Biol. Chem. 273, 16576-16582.
Hutton, M., Busfield, F., Wragg, M., Crook, R., Perez-Tur, J., Clark, R. F., Prihar, Talbot, C., Phillips, H., Wright, K., Baker, M., Lendon, C., Duff, K., Martinez, Houlden, H., Nichols, A., Karran, E., Roberts, G., Roques, P., Rossor, M., Venter, J. C., Adams, M. D., Cline, R. T., Phillips, C. A., Goate, A., et, and AL (1996). Complete analysis of the presenilin 1 gene in early onset Alzheimer's disease. Neuroreport 7, 801-805.
Kehoe, P., Wavrant-De, V. F., Crook, R., Wu, W. S., Holmans, P. Fenton, I., Spurlock, G., Norton, N., Williams, H., Williams, N., Lovestone, S., Perez-Tur, J., Hutton, M., Chartier-Harlin, M. C., Shears, S., Roehl, K., Booth, J., Van Voorst, W., Ramic, D., Williams, J., Goate, A., Hardy, J., and Owen, M. J. (1999). A full genome scan for late onset Alzheimer's disease. Human Molecular Genetics 8, 237-245.
Leitersdorf, E., Hwang, M., and Luskey, K. L. (1990). ScrFI polymorphism in the 2nd intron of the HMGCR gene. Nucleic Acids Res. 18, 5584.
Levy-Lahad, E., Wijsman, E. M., Nemens, E., Anderson, L., Goddard, K. A., Weber, J. L., Bird, T. D., and Schellenberg, G. D. (1995). A familial Alzheimer's disease locus on chromosome 1 [see comments]. Science 269, 970-973.
May, P. C., Johnson, S. A., Poirier, J., Lampert-Etchells, M., Finch, and CE (1989). Altered gene expression in Alzheimer's disease brain tissue. [Review] [41 refs]. Canadian Journal of Neurological Sciences 16, 473-476.
Mills, J. and Reiner, P. B. (1999). Regulation of amyloid precursor protein cleavage. [Review] [240 refs]. JNeurochem, 72, 443-460.
Morgan, K., Morgan, L., Carpenter, K., Lowe, J., Lam, L., Cave, S., Xuereb, J., Wischik, C., Harrington, C., and Kalsheker, N. A. (1997). Microsatellite polymorphism of the alpha 1-antichymotrypsin gene locus associated with sporadic Alzheimer's disease. Hum. Genet. 99, 27-31.
Ness, G. C. and Chambers, C. M. (2000). Feedback and hormonal regulation of hepatic 3-hydroxy-3-methylglutaryl coenzyme A reductase: the concept of cholesterol buffering capacity. Proc Soc. Exp. Biol. Med. 224, 8-19.
Nishiwaki, Y., Kamino, K., Yoshiiwa, A., Sato, N., Tateishi, K., Takeda, M., Kobayashi, T., Yamamoto, H., Nonomura, Y., Yoneda, H., Sakai, T., Imagawa, M., Miki, T, and Ogihara, T. (1997). T/G polymorphism at intron 9 of presenilin 1 gene is associated with, but not responsible for sporadic late-onset Alzheimer's disease in Japanese popuiation. Neurosci. Lett. 227, 123-126.
Okuizumi, K., Onodera, O., Namba, Y., Ikeda, K., Yamamoto, T., Seki, K., Ueki, A., Nanko, S., Tanaka, H., and Takahashi, H. (1995). Genetic association of the very low density lipoprotein (VLDL) receptor gene with sporadic Alzheimer's disease. Nat. Genet. 11, 207-209.
Owen, M. J., Holmans, P., and McGuffin, P. (1997). Association studies in psychiatric genetics [editorial]. Mol. Psychiatry 2, 270-273.
Pericak-Vance, M. A., Bass, M. L., Yamaoka, L. H., Gaskell, P. C., Scott, W. K., Terwedow, H. A., Menold, M. M., Conneally, P. M., Small, G. W., Saunders, A. M., Roses, A. D., and Haines, J. L. (1998). Complete genomic screen in late-onset familial Alzheimer's disease. Neurobiol. Aging 19, S39-S42.
Pericak-Vance, M. A. and Haines, J. L. (1995). Genetic susceptibility to Alzheimer disease. [Review] [66 refs]. Trends in Genetics 11, 504-508.
Poirier, J. (1994). Apolipoprotein E in animal models of CNS injury and in Alzheimer's disease. Trends Neurosci. 17, 525-530.
Poirier, J., Baccichet, A., Dea, D., and Gauthier, S. (1993b). Cholesterol synthesis and lipoprotein reuptake during synaptic remodelling in hippocampus in adult rats. Neuroscience 55, 81-90.
Poirier, J., Davignon, J., Bouthillier, D., Kogan, S., Bertrand, and Gauthier, S. (1993a). Apolipoprotein E polymorphism and Alzheimer's disease [see comments]. Lancet 342, '697-699.
Poirier, J., Delisle, M. C., Quirion, R., Aubert, I., Farlow, M., Lahiri, D., Hui, S., Bertrand, P., Nalbantoglu, J., and Gilfix, B. M. (1995). Apolipoprotein E4 allele as a predictor of cholinergic deficits and treatment outcome in Alzheimer disease. Proceedings of the National Academy of Sciences of the United States of America 92, 12260-12264.
Powell, E. E. and Kroon, P. A. (1992). Measurement of mRNA by quantitative PCR with a nonradioactive label. J. Lipid Res. 33, 609-614.
Reynolds, G. A., Basu, S. K., Osborne, T. F., Chin, D. J., Gil, G., Brown, M. S., Goldstein, J. L., and Luskey, K. L. (1984). HMG CoA reductase: a negatively regulated gene with unusual promoter and 5′ untranslated regions. Cell 38, 275-285.
Reynolds, Goldstein and Brown (1985) Multiple RNAs for Hydroxy-methylcoenzyme A reductase determined by multiple initiation sites in intron splicing in the 5′ untranslated region. J. Biol. Chem. 260: 10369-10377
Rodwell, V. W., Beach, M. J., Bischoff, K. M., Bochar, D. A., Darnay, B. G., Friesen, J. A., Gill, J. F., Hedl, M., Jordan-Starck, T., Kennelly, P. J., Kim, D. Y., and Wang, Y. (2000). 3-hydroxy-3-methylglutaryl-CoA reductase [In Process Citation]. Methods Enzymol. 324, 259-280.
Salen, G., Shefer, S., Batta, A. K., Tint, G. S., Xu, G., Honda, A., Irons, M., and Elias, E. R. (1996). Abnormal cholesterol biosynthesis in the Smith-Lemli-Opitz syndrome. J. Lipid Res. 37, 1169-1180.
Sato, R. and Takano, T. (1995). Regulation of intracellular cholesterol metabolism. Cell Struct. Funct. 20, 421-427.
Schwab, S. G., Bagli, M., Papassotiropoulos, A., Jessen, F., Maier, W., Rao, M. L., and Heun, R. (1999). Alpha-1-antichymotrypsin gene polymorphism and risk for sporadic Alzheimer's disease in a German population. Dement. Geriatr. Cogn Disord. 10, 469-472.
Simons, M., Keller, P., De Strooper, B., Beyreuther, K., Dotti, C. G., and Simons, K. (1998). Cholesterol depletion inhibits the generation of beta-amyloid in hippocampal neurons. Proceedings of the National Academy of Sciences of the United States of America 95, 6460-6464.
Sisodia, S. S., Koo, E. H., Beyreuther, K., Unterbeck, A., and Price, D. L. (1990). Evidence that beta-amyloid protein in Alzheimer's disease is not derived by normal processing. Science 248, 492-495.
Sparks, D. L., Kuo, Y. M., Roher, A., Martin, T., and Lukas, R. J. (2000). Alterations of Alzheimer's disease in the cholesterol-fed rabbit, including vascular inflammation. Preliminary observations. Annals of the New York Academy of Sciences 903, 335-344.
St George-Hyslop, P., Haines, J., Rogaev, E., Mortilla, M., Vaula, G., Pericak-Vance, M., Foncin, J. F., Montesi, M., Bruni, A., and Sorbi, S. (1992). Genetic evidence for a novel familial Alzheimer's disease locus on chromosome 14. Nature Genetics 2, 330-334.
Strittmatter, W. J., Weisgraber, K. H., Huang, D. Y., Dong, L. M., Salvesen, G. S., Pericak-Vance, M., Schmechel, D., 25 Saunders, A. M., Goldgaber, D., and Roses, A. D. (1993). Binding of human apolipoprotein E to synthetic amyloid beta peptide: isoform-specific effects and implications for late-onset Alzheimer disease. Proceedings of the National Academy of Sciences of the United States of America. 90, 8098-8102.
Svennerholm, L., Bostrom, K., Jungbjer, B., and Olsson, L. (1994). Membrane lipids of adult human brain: lipid composition of frontal and temporal lobe in subjects of age 20 to 100 years. J. Neurochem. 63, 1802-1811.
Svennerholm, L. and Gottfries, C. G. (1994). Membrane lipids, selectively diminished in Alzheimer brains, suggest synapse loss as a primary event in early-onset form (type I) and demyelination in late-onset form (type II). J. Neurochem. 62, 1039-1047.
Tysoe, C., Whittaker, J., Cairns, N. J., Atkinson, P. F., Harrington, C. R., Xuereb, J., Wilcock, G., and Rubinsztein, D. C. (1997). Presenilin-1 intron 8 polymorphism is not associated with autopsy-confirmed late-onset Alzheimer's disease. NL 222, 68-69.
Wang, Y., Darnay, B. G., and Rodwell, V. W. (1990). Identification of the principal catalytically important acidic residue of 3-hydroxy-3-methylglutaryl coenzyme A reductase. J. Biol. Chem. 265, 21634-21641.
Wisniewski, T., Golabek, A., Matsubara, E., Ghiso, J., and Frangione (1993). Apolipoprotein E: binding to soluble Alzheimer's beta-amyloid. Biochemical & Biophysical Research Communications 192, 359-365.

1. A method of diagnosing or prognosticating a dementia in a subject, said method comprising:
(a) obtaining a sample from said subject, wherein said sample comprises nucleic acid comprising a 3-hydroxy-3-methylglutaryl coenzyme A reductase (HMGR) gene; and
(b) determining whether said nucleic acid comprises a polymorphism relative to a corresponding control sample obtained from a control subject;
wherein the presence of said polymorphism is used to diagnose or prognosticate a dementia.

2. The method of claim 1, wherein said polymorphism is localized in intron B of said HMGR gene.

3. The method of claim 2, wherein step (b) comprises:
(i) amplifying a nucleic acid sequence comprising said intron B by polymerase chain reaction (PCR) to obtain a PCR product;
(ii) digesting said PCR product with a restriction enzyme to obtain a restriction digest product; and
(iii) determining a size of said restriction digest product.

4. The method of claim 3 wherein said restriction enzyme is ScrFI.

5. The method of claim 1, wherein said sample is a tissue or body fluid of said subject.

6. The method of claim 5, wherein said tissue or body fluid is neural tissue or fluid.

7. The method of claim 5 wherein said tissue or body fluid is selected from saliva, hair, blood, plasma, lymphocytes, cerebrospinal fluid, epithelia and fibroblasts.

8. The method of claim 1, wherein said control subject is a normal age-matched subject.

9. The method of claim 1, wherein the method is used to prognosticate a dementia and wherein the control sample was obtained from the subject at another time.

10. A method of diagnosing or prognosticating a dementia in a subject, said method comprising:
(a) measuring a first level of HMGR activity in a sample obtained from said subject; and
(b) comparing said first level to a second level which is an average HMGR activity measured in at least one corresponding control sample obtained from at least one control subject, whereby if said first level is significantly less than said second level then said subject suffers from a dementia;
wherein said method is used to diagnose or prognosticate a dementia.

11. The method of claim 10, wherein said sample is a tissue or body fluid of said subject.

12. The method of claim 11, wherein said tissue or body fluid is neural tissue or fluid.

13. The method of claim 11 wherein said tissue or body fluid is selected from saliva, hair, blood, plasma, lymphocytes, cerebrospinal fluid, epithelia and fibroblasts.

14. The method of claim 10, wherein said control subject is a normal age-matched subject.

15. The method of claim 10, wherein the method is used to prognosticate a dementia and wherein the control sample was obtained from the subject at another time.

16. A method of diagnosing or prognosticating a dementia in a subject, said method comprising:
(a) obtaining a sample from said subject, wherein said sample comprises ribonucleic acid encoded by an HMGR gene; and
(b) determining whether said sample comprises at least one alteration relative to a corresponding control sample obtained from a control subject, wherein said alteration is selected from the group consisting of:
(i) an increase in a level of a first ribonucleic acid encoded by an HMGR gene, wherein said first ribonucleic acid has a deletion of exon 13;
(ii) an increase in a level of a second ribonucleic encoded by an HMGR gene, wherein said second ribonucleic acid has an insertion of intron M; and
(iii) a decrease in a level of a third ribonucleic acid comprising a normal HMGR transcript;
wherein the presence of said at least one alteration is used to diagnose or prognosticate a dementia.

17. The method of claim 16 wherein said alteration is determined using reverse transcriptase-polymerase chain reaction (RT-PCR).

18. The method of claim 16, wherein said sample is a tissue or body fluid of said subject.

19. The method of claim 18, wherein said tissue or body fluid is neural tissue or fluid.

20. The method of claim 18 wherein said tissue or body fluid is selected from saliva, hair, blood, plasma, lymphocytes, cerebrospinal fluid, epithelia and fibroblasts.

21. The method of claim 16, wherein said control subject is a normal age-matched subject.

22. The method of claim 16, wherein the method is used to prognosticate a dementia and wherein the control sample was obtained from the subject at another time.

23. A commercial package for the diagnosis and/or the prognostication of a dementia, said commercial package comprising at least one of:
(a) means for detecting a polymorphism in an HMGR gene in a sample together with instructions for assessing said polymorphism relative to a corresponding control sample;
(b) means for determining a level of HMGR activity in a sample together with instructions for comparing said level with an established standard or a control level measured in a corresponding control sample; and
(c) means for determining the presence of at least one feature selected from the group consisting of (i) a first HMGR ribonucleic acid having a deletion of exon 13, (ii) a second HMGR ribonucleic acid having an insertion of intron M, (iii) a decrease in a level of a third ribonucleic acid comprising a normal HMGR transcript; and/or an increase in said first and/or second ribonucleic acid relative to said third ribonucleic acid,
together with instructions for comparing said feature with a control feature in a corresponding control sample.

24. The method according to claim 1, wherein said dementia is an Alzheimer disease.

25. The commercial package of claim 23, wherein said dementia is an Alzheimer disease.

26. The method according to claim 10, wherein said dementia is an Alzheimer disease.

27. The method according to claim 16, wherein said dementia is an Alzheimer disease.
